SEQUENCE LISTING (7 sequences)

SEQ ID NO: 1 - DNA, 30 bp, Artificial Sequence
  Description of Artificial Sequence Synthetic primer
ggggaattcagtttccgtcgcggtgagtgg

SEQ ID NO: 2 - DNA, 30 bp, Artificial Sequence
  Description of Artificial Sequence Synthetic primer
gggggatccatgatttccttcggttcgccg

SEQ ID NO: 3 - DNA, 30 bp, Artificial Sequence
  Description of Artificial Sequence Synthetic primer
gggggatcctatgctggctgatgctggcgg

SEQ ID NO: 4 - DNA, 30 bp, Artificial Sequence
  Description of Artificial Sequence Synthetic primer
gggaagctttcaggccgaaagtgccttgaa

SEQ ID NO: 5 - DNA, 42 bp, Artificial Sequence
  Description of Artificial Sequence Synthetic primer
gggaagcttgggtctagaagtttcgctcgcggtgagtggcga

SEQ ID NO: 6 - DNA, 30 bp, Artificial Sequence
  Description of Artificial Sequence Synthetic primer
gggactagttcaggccgaaagtgccttgaa

SEQ ID NO: 7 - protein, 6 aa, Artificial Sequence
  Description of Artificial Sequence Synthetic 6xHis tag
HHHHHH

(19) United States
(12) Patent Application Publication
(10) Pub. No.: US 2012/0202271 A1
(43) Pub. Date: Aug. 9, 2012

(54) Method producing an immune response for reducing the risk of developing brucellosis

(76) Inventors: Gerhardt G. Schurig, Blacksburg, VA (US); Stephen M. Boyle, Blacksburg, VA (US); Nammalwar Sriranganathan, Blacksburg, VA (US)

(21) Appl. No.: 13/385,003

(22) Filed: Jan. 27, 2012

Related U.S. Application Data
(63) Continuation-in-part of application No. 12/589,299, filed on Oct. 21, 2009.

(51) Int. Cl. C12N 1/21; C12N 15/85

(52) U.S. Cl. 435/252.3; 435/320.1

(57) ABSTRACT

This invention relates to unique antigens in a brucellosis vaccine which Provide an immune response against bacteria which cause brucellosis in cattle and Other animals. The vaccine specifically uses Bp26 and LeuB expressed to create the immunity.

This application is a Continuation-In-Part of Ser. No. 12/589,299, filed on Oct. 21, 2009, bearing the same title and with the same inventor and which is currently Pending before the Patent Office

This original application relies upon the provisional application Ser. No. 61/109,804 entitled Producing an Immune Response for Reducing the Risk of Developing Brucellosis filed on Oct. 30, 2008, incorporates it by reference herein, and has the same inventors and the same attorney of record. A copy of that provisional was filed with Ser. No. 12/589,299.

### BACKGROUND

1. Technical Field

This document relates to materials and methods for producing an immune response for reducing the risk of developing brucellosis and other maladies. For example, this invention provides vaccines for administration to animals as well as methods for producing an immune response against bacteria that cause brucellosis using vaccines provided herein.

2. Background Information

Brucellosis is an infectious disease caused by bacteria of the genus Brucella. There are various Brucella species that are capable of infecting both wildlife and livestock. The principal cause of brucellosis in cattle is the bacterium B. abortus. Infected cattle commonly have high incidences of spontaneous abortions, arthritic joints, and retained placenta following calving. In the United States, infected cows are often killed. Sheep and goats are the preferred hosts of B. melitensis, which is the Brucella species most virulent for humans. Humans can become infected by coming in contact with infected animals or animal products, such as unpasteurized milk, that are contaminated with these bacteria.

### SUMMARY

This invention relates to materials and methods for producing an immune response for reducing the risk of developing brucellosis. For example, this invention provides vaccines for administration to animals, both domestic and wildlife, as well as methods for producing an immune response against bacteria that cause brucellosis using vaccines provided herein. The vaccines provided herein can be effective for reducing the risk of developing brucellosis from multiple species of Brucella. For example, isolated DNA constructs, bacteria transformed with DNA constructs (e.g. plasmids), and methods for producing an immune reponse that reduces the risk of developing brucellosis in animals, are provided.

In general, one aspect of this invention features an isolated DNA construct that when expressed in strain RB 51™ for producing an immune response against a bacterium that causes brucellosis. The construct comprises, or consists essentially of a nucleic acid encoding a polypeptide selected from the group consisting of L 7/L 12, WboA Bp26. and 85A. This construct can comprise a nucleic acid encoding more than one of the polypeptides. The construct can comprise a nucleic acid encoding a diagnostic marker protein. The construct can comprise a nucleic acid encoding a L7/L 12 polypeptide, a leuB polypeptide, and a green fluorescent protein (GFP). The construct can comprise a nucleic acid encoding a Bp26 polypeptide, leuB polypeptide, and/or a GFP polypeptide. The construct can comprise a nucleic acid encoding a L7/L12 polypeptide, a Bp26 polypeptide, and a leuB polypeptide. The construct can comprise a nucleic acid encoding a L7/L12 polypeptide, a WboA polypeptide, and a leuB polypeptide. The construct can comprise a nucleic acid encoding a WboA polypeptide, an 85A polypeptide, and a leuB polypeptide.

In another embodiment, the invention features a bacterial cell for producing an immune response against a bacterium that causes brucellosis. The cell comprises, or consists essentially of a nucleic acid encoding a polypeptide selected from the group consisting of L7/L12, WboA, Bp26 or 85A. The bacterium can be Brucella abortus strain RB 51™.

In another embodiment this invention features a method for producing an immune response in an animals against a bacterium that causes brucellosis. The method comprises, or consists essentially of, administering to the animal an amount of bacteria comprising a DNA construct comprising a nucleic acid encoding a polypeptide selected from the group consisting of L7/L12, WboA, and Bp26, under conditions wherein the animal produces antibodies to antigens expressed by the bacteria, thereby producing an immune response against the bacterium that causes brucellosis. The bacteria can be Brucella abortus RB 51™. The animal can be selected from a group consisting of cows, sheep, goats and pigs or other vertebrate species that contracts brucellosis.

Unless otherwise defined, all technical and scientific terms used herein have the same meaning as commonly understood by one of ordinary skill in the art to which this invention pertains. Although methods and materials similar or equivalent to those described herein can be used to practice the invention, suitable methods and materials are described below. All publications, patent applications including provisional applications and other references mentioned herein are incorporated by reference in their entirety. In case of conflict, the present specification, including definitions, will control. In addition, the materials, methods, and examples are illustrative only and not intended to be limiting.

Other features and advantages of the invention will be apparent from the following detailed description and from the claims.

### BRIEF DESCRIPTION OF THE DRAWINGS

FIG. 1 is a vector map of a pLeuB/L7L12/GFP plasmid for expression of a L7L12 polypeptide and a green fluorescent protein (GFP) expression reporter.

FIG. 2 is a vector map of a pLeuB/Bp26/GFP plasmid for expression of a Bp26 polypeptide and a GFP reporter.

FIG. 3 is a vector map of a pLeuB/L7/L12/Bp26 plasmid for expression of a L7/L12 polypeptide and a Bp26 polypeptide.

FIG. 4 is a vector map of a pLeuB/L7L12/WboA plasmid for expression of a L7/L12 polypeptide and a WboA polypeptide.

FIG. 5 is a vector map of a pLeuB/WboA/32 kDa plasmid for expression of an 85A (32 kDa antigen) polypeptide and a WboA polypeptide.

FIG. 6 is a schematic depicting disruption of a leuB gene of B. abortus from an RB 51™ vaccine.

FIG. 7 is a photograph of a Southern blot.

FIG. 8 is a vector map of a pNS4 plasmid for expression of a 3-isopropylmalate dehydrogenase (LeuB) polypeptide for complementation of a leucine auxotrophic B. abortus RB 51™ strain.

FIG. 9 is a plot of colony forming units (log10 CFU) of B. abortus transformed with different DNA constructs per unit time grown in Brucella minimal medium minus leucine.

FIG. 10 is a series of photographs of murine J1771.A1 murine macrophage cells containing B. abortus RB51™ expressing GFP at 36 hours post-infection.

FIG. 11 is a bar graph showing the number of colony forming units (log10CFU) per mouse spleen from mice treated with saline, an RB 51™ vaccine, an RB 51 (TAM) leuB vaccine, a RB 51™ leuB/pNS4 vaccine, or a RB 51™ leuB/pNS4GFP vaccine and challenged with virulent B. abortus 2308.

FIG. 12 is an immunoblot showing reactivity of sera from vaccinated CD-1 mice to purified GFP.

FIGS. 13a and 13b shows Bp26 being over-expressed on a plasmid from either t the TrcD promoter or the Amp signal sequence but without GFP.

### DETAILED DESCRIPTION

This invention relates to materials and methods for producing an immune response for reducing the risk of developing brucellosis. For example, the invention provides vaccines for administration to animals as well as methods for producing an immune response against bacteria that cause brucellosis using vaccines provided herein.

The vaccines provided herein can be in the form of recombinant polypeptides involved in evoking an immune response to bacterium of the genus Brucella, nucleic acid vectors (e.g., plasmids) designed to express such recombinant polypeptides, and bacteria transformed with such nucleic acids. The vaccines provided herein can be used to immunize or treat any type of animal including, without limitation, cows, sheep, goats, pigs, dogs, poultry or any verterbrae species that contracts brucellosis.

The vaccines provided herein can be used to induce an immune response against any species of Brucella including, without limitation, B. abortus, B. canis, B melitensis, B. neotaomae, B. ovis, B. suis and B. pinnipediae. For example, a vaccine provided herein can protect against more than one species of Brucella. In some cases the vaccines provided by this invention can be used to induce an immune response against a pathogen that causes spontaneous abortion in cattle (e.g., Neospora caninum). The vaccines provided can be used to reduce the risk of developing symptoms associated with the disease known as brucellosis.

This invention also provides methods and materials relating to isolated nucleic acid molecules, substantially pure polypeptides, and bacteria that contain an isolated nucleic acid molecule. The term “nucleic acid” as used herein encompasses both RNA and DNA, including cDNA, genomic DNA, and synthetic (e.g., chemically synthesized) DNA. The nucleic acid can be double-stranded or single stranded. Where single-stranded, the nucleic acid can be the sense strand or the antisense strand. In addition, nucleic acid can be circular or linear.

The term “isolated” as used herein with reference to nucleic acid refers to a naturally-occurring nucleic acid that is not immediately contiguous with both of the sequences with which it is immediately contiguous (one of the 5′ end and one on the 3′ end) in the naturally-occurring genome of the organism from which it is derived. For example, an isolated nucleic acid can be, without limitation, a recombinant DNA molecule of any length, provided one of the nucleic acid sequences normally found immediately flanking that recombinant DNA molecule in a naturally-occurring genome is removed or absent. Thus, an isolated nucleic acid includes, without limitation, a recombinant DNA that exists as a separate molecule (e.g., a cDNA or a genomic DNA fragment produced by PCR or restriction endonuclease treatment) independent of other sequences as well as recombinant DNA that is incorporated into a vector, an autonomously replicating plasmid, a virus (e.g., a retrovirus, adenovirus, or herpes virus), or into the genomic DNA of a prokaryote or eukaryote. In addition, an isolated nucleic acid can include a recombinant DNA molecule that is part of a hybrid or fusion nucleic acid sequence.

The term “isolated” as used herein with reference to nucleic acid also includes any non-naturally-occurring nucleic acid since non-naturally-occurring nucleic acid sequences are not found in nature and do not have immediately contiguous sequences in a naturally-occurring genome. For example, non-naturally-occurring nucleic acid such as an engineered nucleic acid is considered to be isolated nucleic acid. Engineered nucleic acid can be made using common molecular cloning or chemical nucleic acid synthesis techniques. Isolated non-naturally-occurring nucleic acid can be independent of other sequences, or incorporated into a vector, an autonomously replicating plasmid, a virus (e.g., a retrovirus, adenovirus, or herpes virus) or the genomic DNA of a prokaryote or eukaryote. In addition, a non-naturally-occurring nucleic acid can include a nucleic acid molecule that is part of a hybrid or fusion nucleic acid sequence.

It will be apparent to those of ordinary skill in the art that a nucleic acid existing among hundreds to millions of other nucleic acid molecules within, for example, cDNA or genomic libraries, or gel slices containing a genomic DNA restriction digest is not to be considered an isolated nucleic acid.

For example, a polypeptide as described herein can be an antigen that produces an immune response in an animal (e.g., antibody production). In some cases a polypeptide provided herein can be expressed as a homologous or heterologous antigen. For example a polypeptide can be an antigen, which is recognized as foreign when expressed in an animal, or a polypeptide can be an enzyme that produces an antigen, which is recognized as foreign when expressed in an animal. For example, a polypeptide provided herein can be an antigen that produces an immune response against Brucella (e.g., a ribosomal protein, an outer membrane protein, a periplasmic protein, or a lipopolysaccharide isolated from a species of Brucella). In some cases a polypeptide for use as described herein can be a L7/L112 polypeptide (e.g., Entrez Gene ID: 3788918), a WboA polypeptide (e.g., Entrez GeneID: 3339782), a Bp26 (e.g., Genbank GI: 32699300) or 85A (32 kDa) polypeptide (e.g. Genbank GI: 894882). In some cases, polypeptide can produce an immune response effective against one species of Brucella and another polypeptide can produce an immune response effective against a different species of Brucella.

In some cases, a vaccine provided herein can be delivered as a prophylactic vaccine to reduce the risk of developing brucellosis should a Brucella infection occur. In some cases, a vaccine provided herein can reduce the risk of developing brucellosis from infection by B. abortus, B. canis, B. melitensis, B. neotomae, B. ovis, B. suis or B. pinnipedia bacteria.

This invention also provides methods for preparing a vaccine provided herein. Such methods can include transforming bacteria with an amount of a nucleic acid vector (e.g., plasmid). Transformation can be achieved by any appropriate method, including, for example, electroporation or chemical transformation.

A vaccine can be produced using an isolated nucleic acid to transform a bacterial culture. For example, a transformed bacterial culture can over-express antigens to produce an immune response. In some cases, an isolated nucleic acid provided herein can include a nucleic acid encoding a L7/L12 polypeptide or a Bp26 polypeptide. In some cases an isolated nucleic acid can include a nucleic acid encoding a 3-isopropylmalate dehydrogenase polypeptide (e.g., leuB,) (e.g., GenBank GI: 62197474). In some cases an isolated nucleic acid can include a nucleic acid that encodes GFP. In some cases, a vaccine provided herein can include a nucleic acid encoding more than one antigen polypeptide (e.g., a L7/L12 polypeptide and a Bp26 polypeptide.). In some cases a vaccine can include a nucleic acid that encodes two, three or four polypeptides.

In some cases, a vaccine provided herein can include a marker of delivery and expression. For example, a vaccine can include a nucleic acid that encodes a flourescent polypeptide (e.g., a GFP) as a marker of expression and delivery of the vaccine to an animal. For example, a marker of delivery and expression can be GFP antibodies in sera from immunized animals.

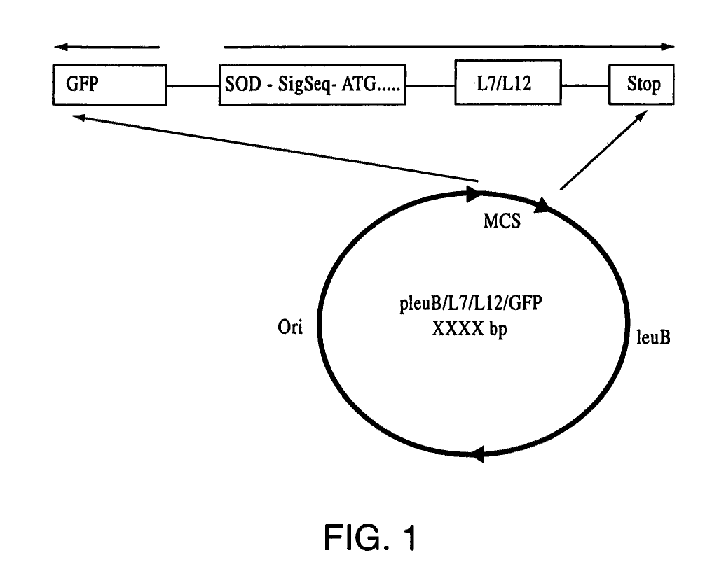

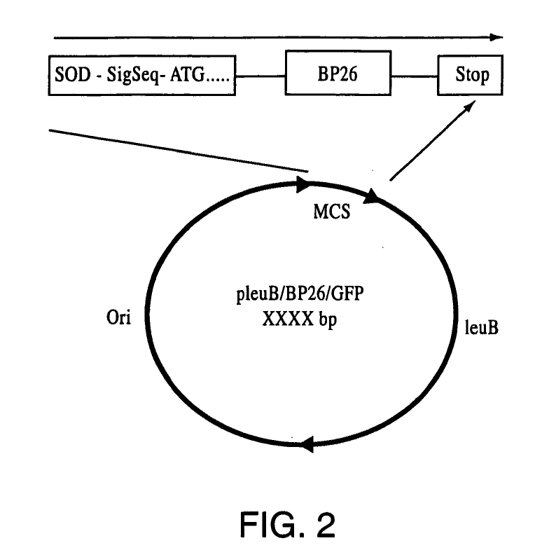

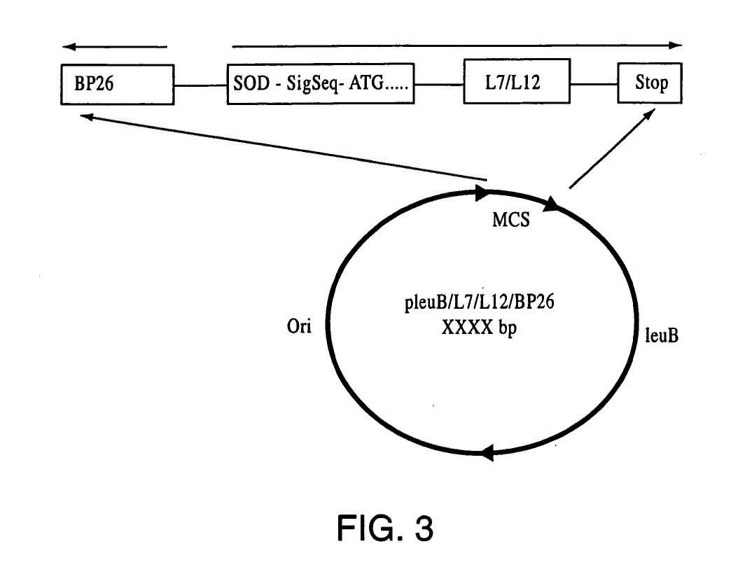

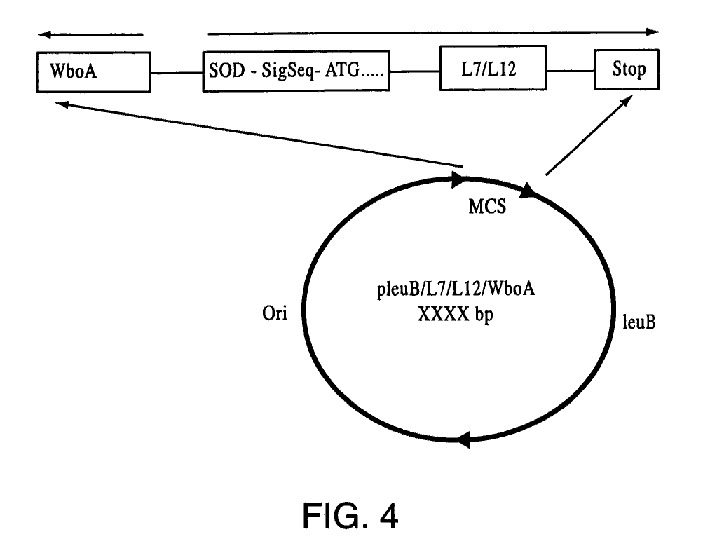

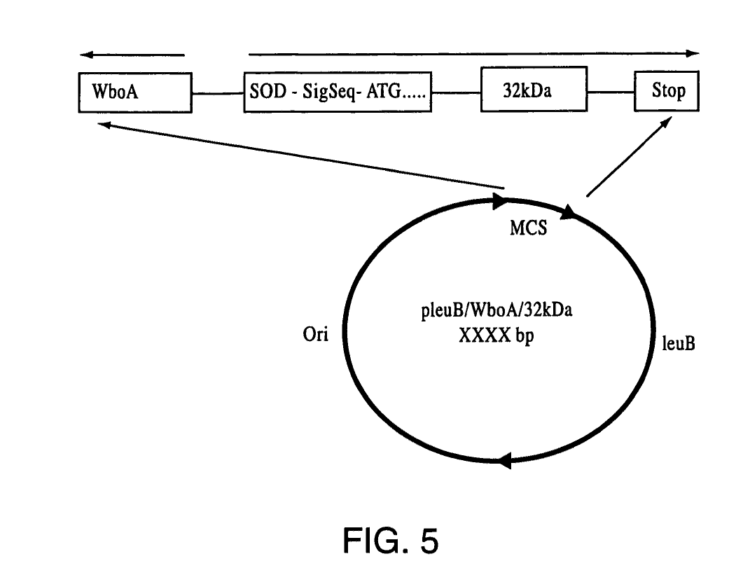

In some cases an isolated nucleic acid provided herein can include a promoter for driving expression of a polypeptide. For example, an isolated nucleic acid can include a nucleic acid encoding a polypeptide operably linked to a promoter sequence. In some cases, a nucleic acid encoding a L7/L12 polypeptide can be operably linked to a SOD promoter sequence (e.g., Ofiate AA, et al, Infect. Immun 67(2): 986-988 (1999). In some cases, an isolated nucleic acid can be transcribed in more than one direction. For example, transcription of a nucleic acid encoding a L7/L12 polypeptide can proceed in a clockwise direction and transcription of a nucleic acid encoding a GFP polypeptide can proceed in a counterclockwise direction. In some cases, an isolated nucleic acid such as a pLeuB/L7/L12GFP plasmid (FIG. 1), a pLeuB/Bp 26/GFP plasmid (FIG. 2) a pLeuB/L7/L12/Bp26 plasmid (FIG. 3), a pLeuB/L7/L12/WboA plasmid (FIG. 4), or a pLeuB/L7/L12/32 kDa plasmid (FIG. 5) can be used to produce a vaccine.

A vaccine for producing an immune response against Brucella can be produced using any bacteria. For example, a bacterial strain such as B. abortus RB 51™ can be used. In some cases, a vaccine can include a strain of bacterium that exhibits leucine auxotrophy. For example, a strain of B. abortus can have a mutation (e.g., deletion) at the leuB locus that disrupts expression of LeuB. In some cases a leucine auxotrophic bacterial strain can be transformed with an isolated nucleic acid to restore leucine biosysthesis. For example, a pleuB/L7/L12/GFP plasmid (FIG. 1), a pLeuB/Bp26/GFP plasmid (FIG. 2), a pLeuB/L7/L12/Bp26 plasmid (FIG. 3), a pLeuB/L7/L12/WboA plasmid (FIG. 4) or a pLeuB/L7/L12/32 kDa plasmid (FIG. 5) can be used to restore leucine biosynthesis in a bacterial strain that exhibits leucine auxotrophy.

The vaccines provided herein can be administered using any appropriate method. Administration can be, for example, topical (e.g. transdermal, ophthalmic or intranasal); pulmonary (e.g., by inhalation or insufflation or powders or aerosols); oral, or parenteral (e.g. by subcutaneous, introthercal, intraventricular, intramuscular, or intraperitoneal injection, or by intravenous drip). Administration can be rapid (e.g., by injection) or can occur over a period of time (e.g., by slow infusion or administration of slow release formulations).

The invention is further described in the following examples, which do not limit the scope of the invention described in the claims.

### EXAMPLES

### Example 1

### Construction of RB 51™ leuB Leucine Auxotroph

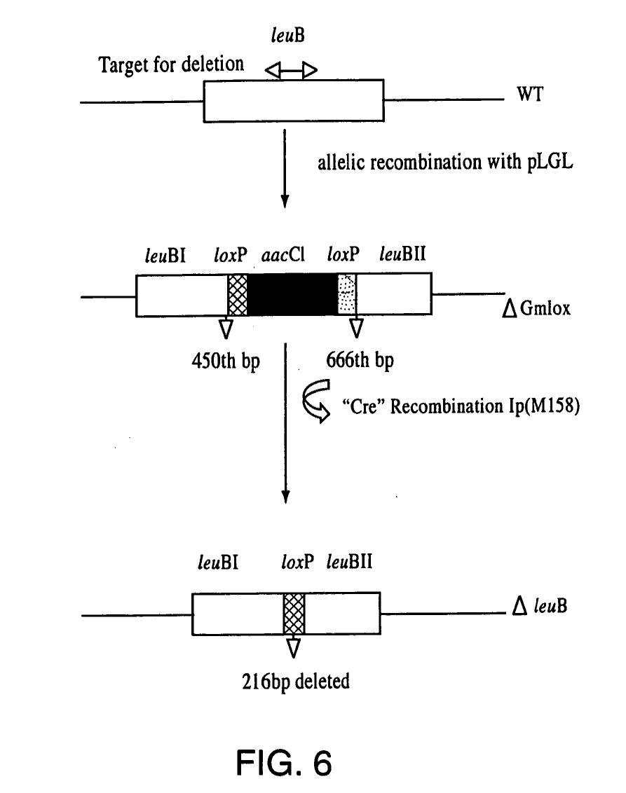

An unmarked mutation was created in the leuB gene of the RB 51™ vaccine strain using cre-lox methodology (FIG. 6). Two regions of the leuB gene of B. abortus RB 51™ were amplified as separate fragments. A 750 bp (leu B1) fragment containing 300 bp upstream of leuB gene was amplified using the primers 5′GGG GAA TTC AGT TTC CGT CGC GGT GAG TGG 3′ and 5′ GGG GGA TCC ATG ATT TCC TTC GGT TCG CCG 3′ and a 450 bp fragment (leu B1 1) was amplified using primer pairs 5′ GGG GGA TCC TAT GCT GGC TGA TGC TGG CGG 3′ and 5; GGG AAG CTT TCA GGC CGA AAG TGC CTT GAA 3′. These two fragments were used to deliberately delete 216 bp of the leuB (FIG. 6) in order to eliminate any reversal of the deletion during subsequent auxotroph growth. The 1200 bp amplicon containing a disrupted leuB gene was cloned into vector pGEM3Z. The acC1 gene coding for gentamicin resistance and flanked by loxP sites was cloned into the BamH1 fragment from the plasmid pUCGmlox.

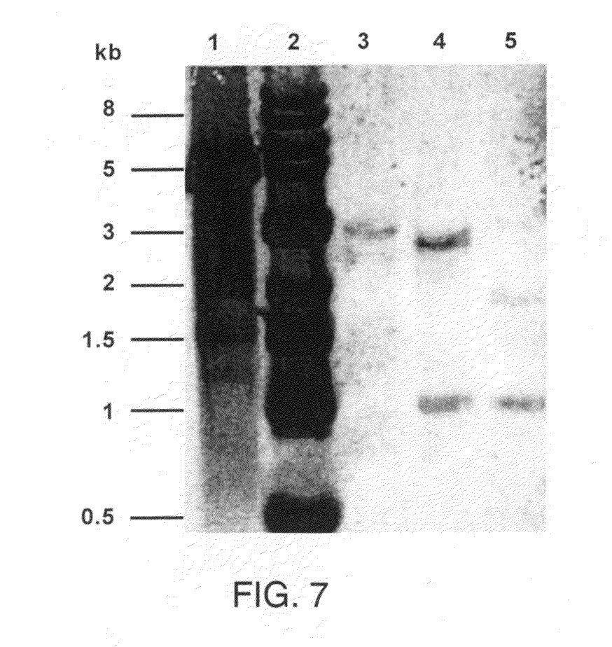

This fragment was cloned into the BamH1 site of the disrupted leuB gene of the intermediate construct to produce the final suicide plasmid pLGL. The suicide plasmid pLGL was introduced into B. abortus RB 51™ by electroportation. The final strain RB 51™ leuB was obtained by plating the transformants on selective medium containing gentamicin and then on medium containing kanamycin. The leucine deficiency of the unmarked mutan was demonstrated in a leucine deficient minimal medium. Southern hybridization was performed to demonstrate that only the leuB gene was disrupted leaving the rest of the B. abortus genome unaltered (FIG. 7). Live B. abortus were handled in a biosafety level 2 facility. Antibiotic resistance markers GmR. AmP, and KnR were used.

### Example 2

### Construction of PNS4/GFP and leuB Complementation

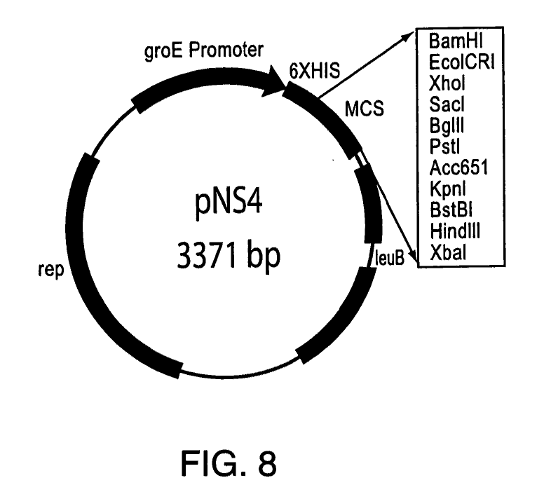

The leu B gene (1412 bp) of vaccine strain RB 51™ along with its promoter was amplified by PCR using the following primers: 5′ GGG-AAG-CTT-GGG-TCT-AGA-AGT-TTC-GCT-CGC-GGT-GAG-TGG-CGA 3′ and 5′ GGG-ACT-AGT-TCA-GGC-CGA-AAG-TGC-CTT-GAA 3′. The origin of replication (1700 bp) and expression cassette (259 bp) with Brucella groE promoter, multiple cloning site, and 6×His tag of plasmid pNSGroE were amplified. After restriction enzyme digestion, the leuB gene fragment and the pNSGroE fragments were purified and ligated to form plasmid pNS4 (FIG. 8). The marker leuB gene was used in place of an antibiotic resistance gene to complement any leuB auxotrophic strains in minimal medium deficient in leucine. Green fluorescent protein (GFP) gene, which was used as a model heterologous antigen, was cloned into the MSC of pNS4 using BamH1 and Xbal sites and designated PNS4/GFP. The complementing plasmid was electroporated into competent RB 51™ leuB, and the transformants were selected by plating on a leucine deficient Brucella minimal medium (BMM) plates. The complemented RB 51™ leuB expressing GFP appeared as green flourescent colonies when observed under UV light, which were later screened for prescence of pNS4/GFP by plasmid extraction and restriction mapping. The expression of GFP was confirmed by immunoblot using GFP antibodies.

### Example 3

### In vitro Growth

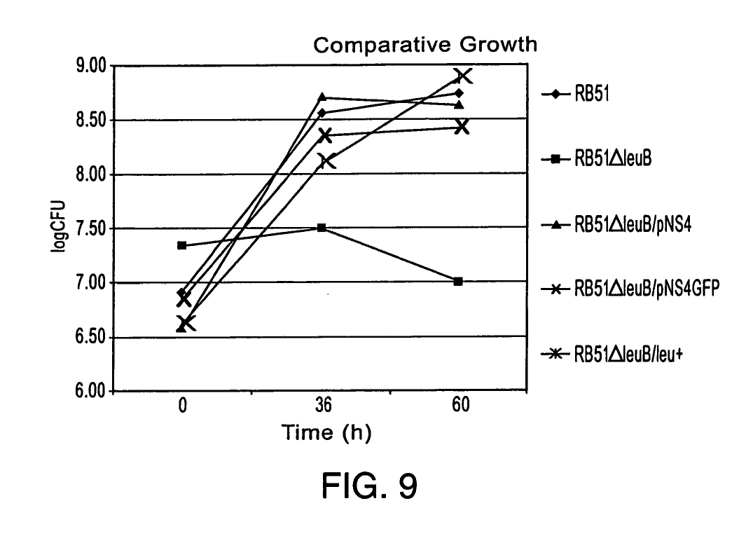

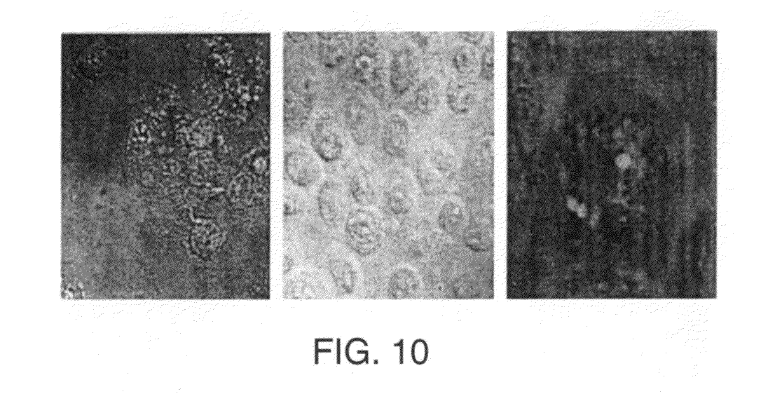

A single colony of a particular B. abortus leuB clone was inoculated in liquid BMM and grown for 72 hours at 37 degrees Centigrade and 200 rpm to create a starter culture. The starter culture was used to inoculate the minimal medium and adjusted to 10-12 Klett Units (KU). At different time points the KU were measured using a Klett-Summerson colorimeter, and corresponding colony forming units (CFUs) were determined. The doubling time in minimal media of RB 51™ or the complemented leuB auxotrophs was observed to be about 7 hours Leucine deficient BMM did not support the growth of the leuB auxotrophs. Complementatic of the leuB auxotrophs with PNS4 restored their growth in leucine deficient BMM. Expression of GFP in pNS4/GFP transformed B. abortus did not affect the strain's ability to be complemented with leuB (FIG. 9)

### Example 4

### Expression of GFP in Infected Macrophages

Murine J774.A1 macrophage cells were plated on a 6 well plate to make them adherent on cover slips and incubated in Dulbecco's Minimal Essential Medium (DMEM) containing 10% fetal bovine serum (FBS) for 24 hours at 37 degrees C. in 5% CO2. A 48 hour culture of the pNS4/GFP complemented RB 51™ leuB transformed B. abortus was re-suspended in PBS and used to infect the macrophages at 100:1 multiplicity of infection (bacteria:macrophage). After 45 minutes the macrophages were washed 3 times with PBS and then incubated in DMEM containing 100 ug/ml. of Steptomycin-penicillin. At 24, 36 and 72 hours post-infection, the cover slips were washed, fixed in formalin, and mounted on glass slides. The slides were observed under a Zeiss LSM at 510 nm. laser scanning microscope in fluorescent mode to detect expression of GFP. J774.A1 macrophages infected with B. abortus containing the leuB complementing plasmid pNS4/GFP appeared green when viewed via 510 nm excitation. FIG. 5 is a representative picture of macrophages containing B. abortus expressing GFP at 36 hours post-infection.

### Example 5

### Immunization of Mice with RB 51™/leuB and pNS4 Strains of Brucella

The protective efficiacy of the B. abortus RB51™/leuB and the leuB-complemented strain were evaluated using 5 to 6 week old female CD-1 mice. The CD-1 mice were used because the mice are from an outbred strain that more closely modeled outbred genetic backgrounds subjected to vaccination under field conditions, e.g., cattle. Four groups of 10 mice were vaccinated intraperitoneally with 3−5×10 8^{th} CFU in 100 ul. with strain RB 51™, RB 51™ leuB/pNS4 or Rb51™ leuB/pNS4/GSP. Another group of 10 mice were bled 5 weeks post-vaccination for harvesting serum. The sera were screened for GFP specific antibodies by immunoblot. At 6 weeks post vaccination, all groups of mice were challenged intraperitoneally with 4×10 8^{th} CFU of B. abortus strain 2308. At two weeks post challenge, mice were euthanized by CO2 asphyxiation and the spleens recovered. The spleens were homogenized, serially diluted and plated on TSA plates to estimate CFUs. The leuB auxotroph and the complemented leuB auxotroph of strain RB 51™ were able to protect the CD-1 mice against a virulent B. abortus strain 2308 challenge (FIG. 6). There was no significant difference in the protection levels (i.e., splenic clearance) afforded by the leuB auxotroph, when the complemented leuB auxotroph and the complemented leuB auxotroph expressing GFP were compared to the mice vaccinated with the strain RB 51™. There was, however, a significant difference in protection afforded between the mice vaccinated with any of the RB51™ strains and the saline control ((P<0.005). Only sera from the group inoculated with RB51™ leuB/pNS4/GFP possessed GFP specific antibodies (FIG. 7). In a separate experiment, the leuB auxotroph and the complemented leuB auxotroph were cleared within 4 to 5 weeks from CD-1 mouse spleens. The rate of clearance was the same as observed with the vaccine RB51™ The splenic clearance of B. abortus strain 2308 was analyzed to determine of variance. The means and variances were compared using Tukey's method.

### Example 6

### Over-Expression of Bp26 is being Over-Expressed without GFP

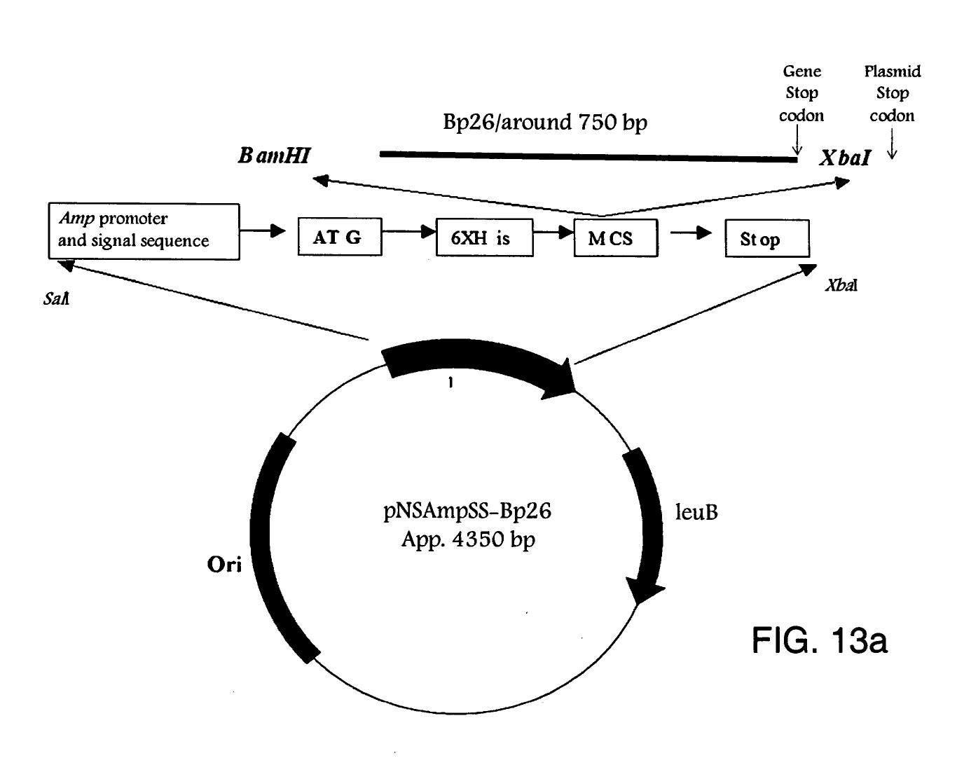

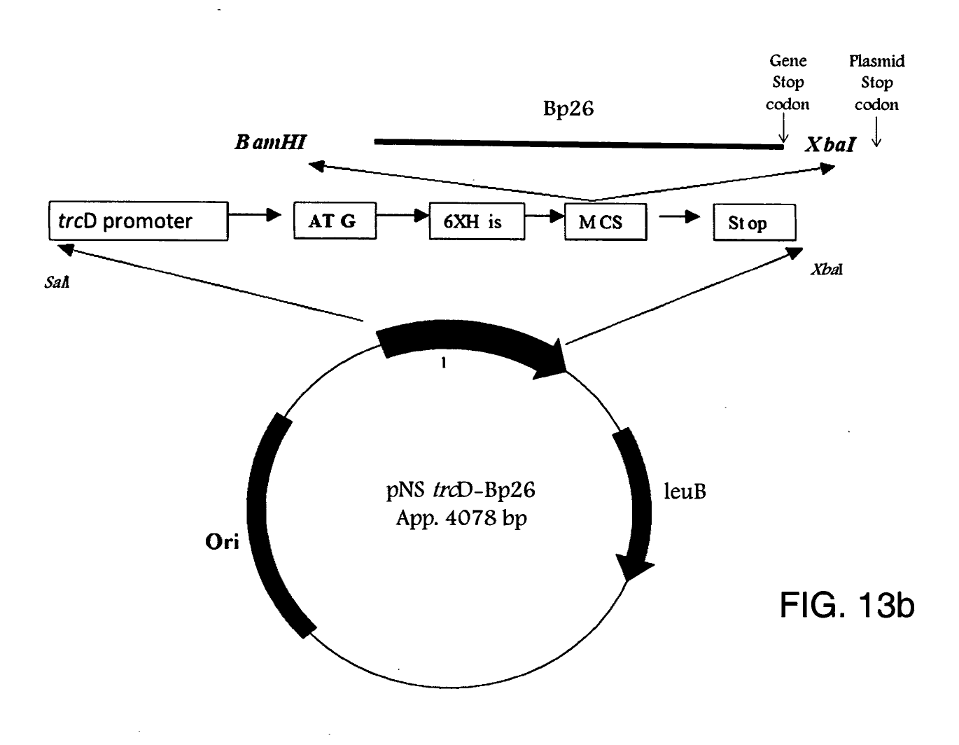

Example 6 is the over-expression of Bp26 without GFP. The two BP26 over-expressing strains B. abortus RB51 pNSTrcD-Bp26 and RB51 PNSAmpSS-Bp26 (FIGS. 13a. and 13b.) provided excellent level of protection in out bred 6 to 8 week-old CD1 mice, when challenged with a 4 by 10(5) cfu wild type B. melitensis 16M. The following table shows the details. Note that the challenge does in this experiement was 10 fold higher than the normal challenge does of 4×10(5) cfu for B. melitensis 16M.

| Vaccine | cfu/spleen | SD | Log protection |
|---|---|---|---|
| RB51AmpSS-Bp26 | 2.11 | 1.35 | 3.12** |
| RB51TrcD-BP26 | 2.43 | 1.28 | 2.80** |
| RB51 | 4.39 | 0.77 | 0.84* |
| Saline group | 5.23 | 0.40 | 0.00 |

*Significant at p < 0.05 and
**Highly significant at p < 0.01

Table Legend: Clearance study—6 to 8 weeks old male CD1 mice were divided into 4 groups. First group (20 mice) was inoculated intraperitoneally (I.P.) with 6.2×10⁸ CFU of RB51 AmpSS-BP26. The second group (20 mice) was inoculated I. P. with 6×10⁸ CFU of B. abortus strain RB51TrcD-BP26 per mouse. The third group (20 mice) was inoculated I. P. with 5.6×10⁸ CFU of strain RB51. The fourth group (20 mice) was inoculated I. P. with saline. Six weeks after immunization these mice were challenged I. P. with 0.2 ml containing 6.4×10⁵ CFU of virulent B. melitensis 16 M per mouse. Two weeks after challenge infection, all the mice were killed by CO2 asphyxiation and the spleens were harvested aseptically and placed in sterile Petri dishes made in TSB and were plated on TSA plates and incubated at 37° C. in the presence of 5% Co2 for 5 days to determine the CFU per spleen, i.e., the clearance of B. melitensis 16M.

As compared to its parent plasmid pBBR1MCS, the pNS plasmids were more stable in B. abortus under non-selective conditions in vitro and in vivo. Even after 11 sub-cultures in an enriched media (non-selective conditions) it was possible to recover the plasmid pNS4 from 10 random colonies of leuB deficient strains of B. abortus. This suggests that the complementing plasmid pNS4 was stable inside the auxotroph under leucine sufficient conditions and that the plasmid was expressed in both selective and non-selective conditions. When viewed under ultraviolet light, all the colonies of RB51™/LEUb transformed with pNS/GroE/::GFP{and those colonies recovered from CD-1 mouse spleens displayed green fluorescence. Combined with the GFP expression noted in macrophages, these data suggest the pNS4 was able to express a heterologous antigen (GFP) in B. abortus following immunization of mice.

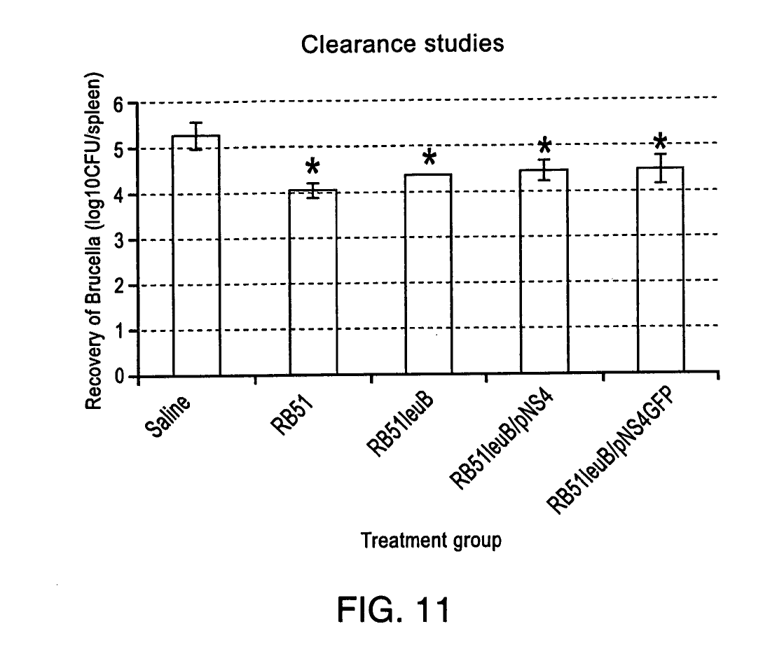

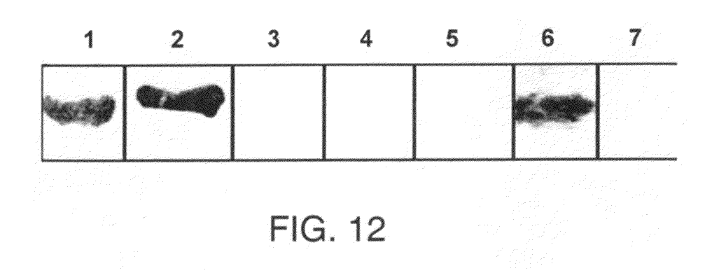

The protective efficacy of the RB51™ leuB vaccine and the leuB-complemented versions in CD-1 mice was as good as found for RB 51™ vaccine in BALB/c mice (FIG. 11). Both the leucine auxotroph and the complemented version of RB51™ vaccine were cleared in CD-1 mice at the same rate as they were in inbred BALB/c mice. The leuB gene appears to provide selective pressure to retain pNS4 when B. abortus is grown in a nutrient limited environment. Immunoblot using purified GFP specific antibody response (FIG. 12). These results suggest that protective homologous or heterologous antigens can replace the GFP encoding nucleic acid and that the over-expression driven by RB51™ leuB can induce a protective response against B. abortus and other infectious agents.

In general this invention provides an RB51™ vaccine that will protect cattle against challenge with B. abortus, and against infection with Mycobacterium paratuberculosis. The vaccine will not contain any new antibiotic resistance than the original resistance in RB51™ (rifampicin) and therefore will not have any objections for approval by the authorities regarding antibiotic resistance. The vaccine will overexpress 1 homologous antigen, cytoplasmic O-chain, and will express one heterologous antigen, 32 Kda protein from M, paratuberculosis utilizing a leucine auxotroph of vaccine M. abortus strain RB51™ strain complemented by a plasmid expressing a leuB gene and the genes for homologous Brucella “O” chain antigen and heterologous mycobacterial 32 kDa antigen. The vaccine is unique as it is an improvement of the approved and tested RB 51™ vaccine and will protect against multiple species of Brucella (B. abortus, B melitensis, and B. suis), will give higher level of protection than existing vaccines due to homologous over-expression of protective Brucella antigens and will confer protection against infection with M. paratuberculosis. In addition the vaccine will not carry any new drug resistant characteristics different from strain RB 51™.

### Other Embodiments

It is to be understood that while the invention has been described in conjunction with the detailed description thereof, the foregoing description is intended to illustrate and not limit the scope of the invention, which is defined by the scope of the appended claims. Other aspects, advantages and modification may be made by those of ordinary skill in the art but are within the scope of the following claims.

What is claimed is:

1. An isolated DNA construct for producing an immune response against a bacterium that causes brucellosis, said construct comprising a nucleic acid encoding a polypeptide selected from the group consisting of LeuB and Bp26.

2. The DNA construct of claim 1, wherein said construct comprises a nucleic acid encoding more than one of said polypeptides.

3. The DNA construct of claim 1 wherein said construct comprises a nucleic acid encoding an expression reporter.

4. The DNA construct of claim 1, wherein said construct comprises a nucleic acid encoding a leuB polypeptide.

5. The DNA construct of claim 1, where said construct comprises a nucleic acid encoding a Bp26 polypeptide and a leuB polypeptide.

6. The DNA construct of claim 1 wherein said construct comprises a nucleic acid encoding a Bp26 polypeptide and a leuB polypeptide and a GFP polypeptide.

7. The DNA construct of claim 1 wherein said construct comprises a nucleic acid encoding at least a Bp26 polypeptide.

8. The DNA construct of claim 1 wherein said construct comprises a nucleic acid encoding a wboA polypeptide and a leuB polypeptide.

9. A bacterial cell for producing an immune response against a bacterium that causes brucellosis, said cell comprising a nucleic acid encoding a polypeptide selected from the group consisting of LeuB, wboA, Bp26 and GFP

10. A bacterial cell of claim 9, wherein said bacterium is Brucella abortus strain RB51.

11. An isolated DNA construct for producing an immune response against a bacterium that causes brucellosis, said construct comprising a nucleic acid encoding a polypeptide from the group including LeuB.

12. An isolated DNA construct for producing an immune response against a bacterium that causes brucellosis, said construct comprising a nucleic acid encoding a polypeptide from the group including Bp26.

13. An isolated DNA construct as in claim 12 wherein the overexpression of Bp26 is RB 51 pNSTrcD-Bp26.

14. An isolated DNA construct as in claim 12 wherein the overexpression of Bp26 is RB 51 pNSAmpSS-Bp26.
